(19) United States
(12) Patent Application Publication
(10) Pub. No.: US 2011/0160250 A1
(43) Pub. Date: Jun. 30, 2011

(54) CRYSTALLINE FORMS OF A FACTOR XA INHIBITOR

(75) Inventors: Anjali Pandey, Fremont, CA (US); Juan Wang, Foster City, CA (US); Joseph Lambing, Burlingame, CA (US)

(73) Assignee: Millennium Pharmaceuticals, Inc.

(21) Appl. No.: 12/970,818

(22) Filed: Dec. 16, 2010

Related U.S. Application Data
(60) Provisional application No. 61/287,681, filed on Dec. 17, 2009.

(51) Int. Cl. A61K 31/4439; C07D 409/14; C12N 9/99; A61P 9/10; A61P 7/02

(52) U.S. Cl. 514/341; 546/275.1; 435/184

(57) ABSTRACT

The present invention provides crystalline forms of a mesylate salt of the compound 5-chloro-N-((1-(4-(2-oxopyridin-1(2H)-yl)phenyl)-1H-imidazol-4-yl)methyl)thiophene-2-carboxamide and pharmaceutical compositions and methods thereof.

### CROSS REFERENCE TO RELATED APPLICATIONS

This application claims the benefit under 35 U.S.C. §119(e) of U.S. Provisional Application Ser. No. 61/287,681 filed on Dec. 17, 2009, which is hereby incorporated by reference in its entirety.

### BACKGROUND OF THE INVENTION

1. Field of the Invention

This invention is directed to crystalline forms of a mesylate salt of a factor Xa inhibitor 5-chloro-N-((1-(4-(2-oxopyridin-1(2H)-yl)phenyl)-1H-imidazol-4-yl)methyl)thiophene-2-carboxamide and the pharmaceutical compositions and uses thereof.

2. State of the Art

5-chloro-N-((1-(4-(2-oxopyridin-1(2H)-yl)phenyl)-1H-imidazol-4-yl)methyl)thiophene-2-carboxamide, having the formula of:

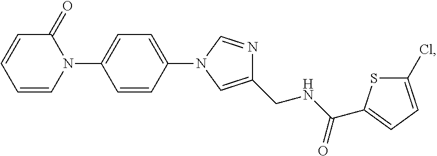

(collectively referred to as “Compound I” herein), is a factor Xa inhibitor described in U.S. Pat. Nos. 7,763,608 and 7,767,697 (which are incorporated by reference in their entirety) and has been shown to have anticoagulation activity in vivo.

### SUMMARY OF THE INVENTION

In one aspect, this invention provides the mesylate salt of 5-chloro-N-((1-(4-(2-oxopyridin-1(2H)-yl)phenyl)-1H-imidazol-4-yl)methyl)thiophene-2-carboxamide, having the formula of:

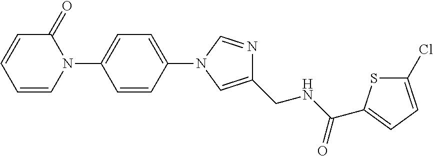

(also referred to as Compound I), which crystalline form is
(a) a crystalline Form C, which is characterized by an X-ray powder diffraction (XRPD) pattern substantially the same as an X-ray powder diffraction (XRPD) pattern having at least six 2θ° peaks selected from the group consisting of about 8.8, about 10.6, about 13.8, about 17.1, about 18.5, about 21.3, about 23.5, about 25.2, about 27.9 and about 29.0; or
(b) a crystalline Form D, which is characterized by an X-ray powder diffraction (XRPD) pattern substantially the same as an X-ray powder diffraction (XRPD) pattern having at least six 2θ° peaks selected from the group consisting of about 8.5, about 10.4, about 16.6, about 17.1, about 17.8, about 22.7, about 23.5, about 24.5, about 26.9 and about 27.5.

In some embodiments, this invention provides a crystalline form characterized by an XRPD pattern as provided in FIG. 3.

In some embodiments, this invention provides a crystalline form characterized by an XRPD pattern as provided in FIG. 4.

In one aspect, this invention provides a mesylate salt wherein at least a portion of the salt is present in a crystalline form wherein the crystalline form is Form C or Form D.

In another aspect, this invention provides a pharmaceutical composition comprising a pharmaceutically acceptable excipient and a therapeutically effective amount of a crystalline form of the mesylate salt of Compound I wherein the crystalline form is Form C or Form D.

In another aspect, this invention provides a composition which is a free-flowing dosable aqueous suspension, comprising a pharmaceutically acceptable aqueous carrier and a mesylate salt of the compound of 5-chloro-N-((1-(4-(2-oxopyridin-1(2H)-yl)phenyl)-1H-imidazol-4-yl)methyl)thiophene-2-carboxamide; wherein the concentration of the compound is at least 40 mg/mL, and wherein at least a portion of the salt is:

(a) in a crystalline form characterized by an X-ray powder diffraction (XRPD) pattern having at least six 2θ° peaks selected from the group consisting of about 8.8, about 10.6, about 13.8, about 17.1, about 18.5, about 21.3, about 23.5, about 25.2, about 27.9 and about 29.0; or
(b) in a crystalline form characterized by an XRPD pattern having at least six 2θ° peaks selected from the group consisting of about 8.5, about 10.4, about 16.6, about 17.1, about 17.8, about 22.7, about 23.5, about 24.5, about 26.9 and about 27.5; or
(c) in an amorphous form; or combinations thereof.

In some embodiments, the crystalline form is characterized by an XRPD pattern substantially the XRPD as FIG. 3.

In some embodiments, the crystalline form is characterized by an XRPD pattern substantially the XRPD as FIG. 4.

In another aspect, this invention provides a method for preventing or treating a condition in a mammal characterized by undesired thrombosis comprising administering to said mammal a therapeutically effective amount of a crystalline form of a mesylate salt of Compound I wherein the crystalline form is Form C or Form D.

In another aspect, this invention provides a method of preparing the a free-flowing composition, comprising mixing the mesylate salt of 5-chloro-N-((1-(4-(2-oxopyridin-1(2H)-yl)phenyl)-1H-imidazol-4-yl)methyl)thiophene-2-carboxamide in an amount of at least 40 mg/mL in a pharmaceutically acceptable aqueous carrier to form a mixture, and storing the mixture at a temperature of about or below 8° C. for a period of time sufficient to form the composition.

This and other embodiments will be further described in the text that follows.

### BRIEF DESCRIPTION OF THE DRAWINGS

FIG. 1 provides an X-ray powder diffraction (XRPD) pattern of crystalline Form A of the mesylate salt of Compound I.

FIG. 2 provides an XRPD pattern of crystalline Form B of the mesylate salt of Compound I.

FIG. 3 provides an XRPD pattern of crystalline Form C particles of the mesylate salt of Compound I isolated from aqueous slurries of Compound I mesylate form A after refrigerated storage overnight.

FIG. 4 provides an XRPD pattern of crystalline Form D particles of the mesylate salt of Compound I isolated from aqueous slurries of Compound I mesylate form A after multiple cycles of −20° C. freezing and thawing during dosing formulation toxicity studies.

FIG. 5 provides an XRPD pattern of crystalline Form E particles (needle shaped) of the mesylate salt of Compound I isolated from aqueous slurries of Compound I mesylate Form B after overnight storage at room temperature.

FIG. 6 provides an XRPD pattern of amorphous particles isolated from aqueous slurries of Compound I mesylate form A after storage at −80° C. overnight and thawed.

FIG. 7 provides an XRPD pattern of a free base of Compound I.

### DETAILED DESCRIPTION OF THE INVENTION

As used herein, the following definitions shall apply unless otherwise indicated.

### Definitions

As used in the specification and claims, the singular form “a”, “an” and “the” include plural references unless the context clearly dictates otherwise.

As used herein, the term “comprising” is intended to mean that the compositions and methods include the recited elements, but not excluding others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of other ingredients and substantial method steps recited. Embodiments defined by each of these transition terms are within the scope of this invention.

The term “Compound I” refers to the compound 5-chloro-N-((1-(4-(2-oxopyridin-1(2H)-yl)phenyl)-1H-imidazol-4-yl)methyl)thiophene-2-carboxamide, having the structure:

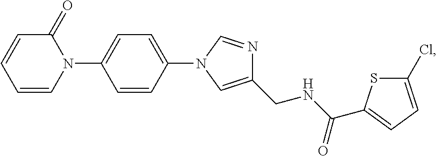

which is described in U.S. Pat. Nos. 7,763,608 and 7,767,697.

“Tautomer” refers to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles. For example, the proton of a salt of Compound I may be in different positions of a imidazole ring the molecule. A salt of Compound I includes all structural variations due to the position of the salt proton unless otherwise indicated.

“Patient” refers to mammals and includes humans and non-human mammals.

“Amorphous” refers to a composition comprising a compound that contains no or too little crystalline content of the compound to yield a discernable pattern by XRPD or other diffraction techniques. For example, glassy materials are a type of amorphous material. Amorphous materials do not have a true crystal lattice, and are glassy rather than true solids, technically resembling very viscous non-crystalline liquids. Glasses may better be described as quasi-solid amorphous material. As is known in the art, an amorphous material refers to a quasi-solid, glassy material. A compound in an amorphous state may be produced by rapidly evaporating solvent from a solvated compound, or by grinding, pulverizing or otherwise physically pressurizing or abrading the compound while in a crystalline state.

“Crystalline” refers to a material that contains a specific compound or a salt of the compound, which may be hydrated and/or solvated, and has sufficient crystalline content to exhibit a discernable diffraction pattern by XRPD or other diffraction techniques, DSC, TGA, stability, solubility, etc. The X-ray diffraction pattern is presented by characteristic 2θ° peaks. One skilled in the art can readily identify a crystalline form of a compound or a salt based on the characteristic 2θ° peaks of an X-ray diffraction pattern of the polymorph. When two X-ray diffraction patterns have at least 4, or at least 6, 8, or 10 2θ° peaks, or all peaks, that do not vary more than ±0.2, ±0.1±0.05 or ±0.02 degrees, it is deemed that the X-ray diffraction patterns are substantially the same. The different polymorphic forms of the same compound can have an impact on one or more physical properties, such as stability, solubility, melting point, bulk density, flow properties, bioavailability, etc.

In some cases, a crystalline material that is obtained by direct crystallization of a compound dissolved in a solvent or solvent mixture or solution or interconversion of crystals obtained under different crystallization conditions, may have crystals that contain the solvent used in the crystallization. Such compositions may be referred to as a crystalline solvate. When the solvent is water, such compositions may be referred to as a crystalline hydrate. Also, the specific solvent system and physical embodiment in which the crystallization is performed, collectively termed as crystallization conditions, may result in the crystalline material having physical and chemical properties that are unique to the crystallization conditions. This may be due to the orientation of the chemical moieties of the compound with respect to each other within the crystal and/or the predominance of a specific polymorphic or pseudopolymorphic form of the compound in the crystalline material. General methods for precipitating and crystallizing a compound may be applied to prepare the various polymorphs or pseudopolymorphs described herein. These general methods are known to one skilled in the art of synthetic organic chemistry and pharmaceutical formulation, and are described, for example, by J. March, “Advanced Organic Chemistry: Reactions, Mechanisms and Structure,” 4th Ed. (New York: Wiley-InterScience, 1992) and Remington: The Science and Practice of Pharmacy, A. Gennaro, ed., 21st edition, -Lippincott, Williams & Wilkins, Philadelphia, Pa., 2006.

“Polymorph” or “polymorphic form” refers to a crystalline form of a substance that is distinct from another crystalline form but that shares the same chemical formula.

“Pseudopolymorph” refers to a crystalline form of a hydrate or solvate of a compound. In contrast to polymorphs, pseudopolymorphs are chemically identical except differ in the amount of water or solvent bound in the crystal lattice. Depending on the solvent used during synthesis and/or crystallization some compounds form hydrates (with water) or solvates (with other solvents) in different stoichiometric ratio. Pseudopolymorphs may show different physical properties like habitus, stability, dissolution rate and bioavailability as known for polymorphs.

“Flowability” refers to the capability of a liquid or loose particulate solid to move by flow and can be determined by methods known in the art.

“Free-flowing dosable aqueous suspension” refers to a composition comprising an aqueous carrier and particles of a polymorph of this invention suspended in the aqueous carrier, where the particles substantially do not adhere to one another and where the volume of the composition can be readily and accurately measured to provide a desired dosage.

It is to be understood that when a value is recited for a condition or a yield, the value may vary within a reasonable range, such as ±5%, ±1%, and ±0.2%. Similarly, the term “about” when used before a numerical value indicates that the value may vary within reasonable range, such as ±5%, ±1%, and ±0.2%. When about is used before a 2θ° peak of an XRPD, it indicates that the 2θ° value may vary ±0.2, ±0.1, ±0.05 or ±0.02 degrees.

“Treatment” or “treating” means any treatment of a disease or disorder in a subject, including:

preventing or protecting against the disease or disorder, that is, causing the clinical symptoms not to develop;

inhibiting the disease or disorder, that is, arresting or suppressing the development of clinical symptoms; and/or

relieving the disease or disorder that is, causing the regression of clinical symptoms. As used herein, the term “preventing” refers to the prophylactic treatment of a patient in need thereof. The prophylactic treatment can be accomplished by providing an appropriate dose of a therapeutic agent to a subject at risk of suffering from an ailment, thereby substantially averting onset of the ailment.

It will be understood by those skilled in the art that in human medicine, it is not always possible to distinguish between “preventing” and “suppressing” since the ultimate inductive event or events may be unknown, latent, or the patient is not ascertained until well after the occurrence of the event or events. Therefore, as used herein the term “prophylaxis” is intended as an element of “treatment” to encompass both “preventing” and “suppressing” as defined herein. The term “protection,” as used herein, is meant to include “prophylaxis.”

“Therapeutically effective amount” refers to that amount of a compound of this invention that is sufficient to effect treatment, as defined below, when administered to a subject in need of such treatment. The therapeutically effective amount will vary depending upon the subject and disease condition being treated, the weight and age of the subject, the severity of the disease condition, the particular compound chosen, the dosing regimen to be followed, timing of administration, the manner of administration and the like, all of which can readily be determined by one of ordinary skill in the art.

“Disease condition” refers to a disease state for which the compounds, compositions and methods of the present invention are being used against.

“Blood sample” refers to whole blood taken from a subject, or any fractions of blood including plasma or serum.

### Polymorphs and Pseudopolymorphs

In one aspect, this invention provides certain polymorphs and/or pseudopolymorphs of the mesylate salt of Compound I.

“The mesylate salt of Compound I” refers the salt formed between Compound I and methanesulfonic acid (CH₃SO₃H), preferably in an equivalence ratio of about 1 to 1.

In some embodiments, the mesylate salt of Compound I is of the formula:

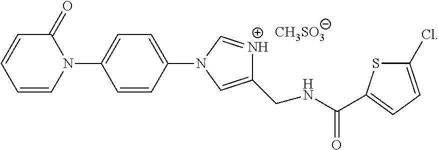

A crystalline polymorph of a given compound is chemically identical to any other crystalline polymorph of that compound in containing the same atoms bonded to one another in the same way, but differs in its crystal forms. Pseudopolymorphs are chemically identical except differ in the amount of water or solvent bound in the crystal lattice. Depending on the solvent used during synthesis and/or crystallization some compounds form hydrates (with water) or solvates (with other solvents) in different stoichiometric ratio. The different crystalline forms of the same compound can have an impact on one or more physical properties, such as stability, solubility, melting point, bulk density, flow properties, bioavailability, etc.

Polymorphs and pseudopolymorphs can be characterized by their crystalline structure (as determined by X-ray diffraction pattern (XRPD)), their thermal properties, as determined by differential scanning calorimetry (DSC) and thermal gravimetric analysis (TGA), stability, solubility, etc. A compound described herein may also form hydrate crystalline forms comprising the compound or a salt thereof and water molecules.

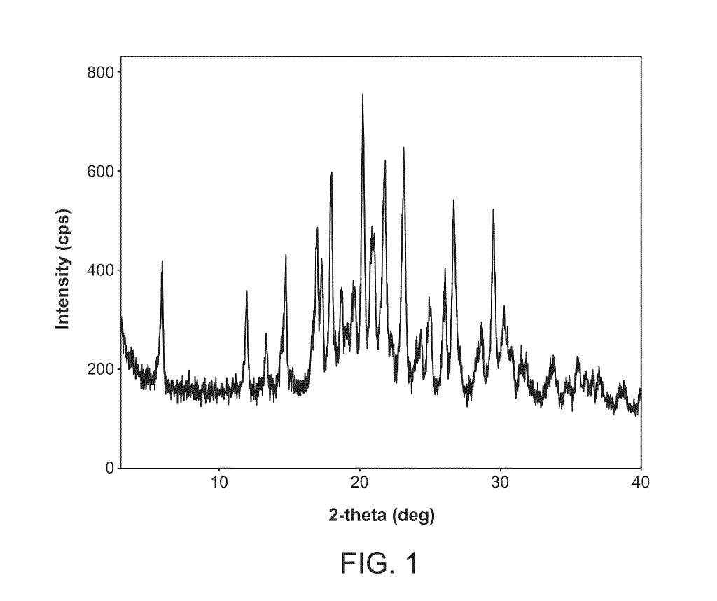

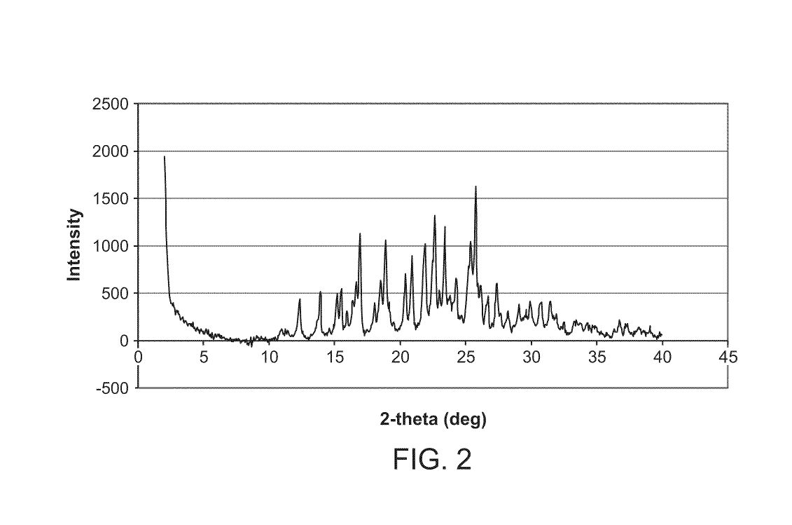

U.S. Provisional Patent Application No. 61/287,683, filed Dec. 17, 2009, and U.S. patent application Ser. No. 12/______, attorney docket number 070545-2001, filed on Dec. 16, 2010, both of which are titled “Salts and Crystalline Forms of a Factor Xa Inhibitor” and incorporated herein by reference in their entirety, describe additional crystalline Forms A and B of the mesylate salt of Compound I, whose XRPD are shown in FIGS. 1 and 2. This invention provides three crystalline forms, Form C, Form D and Form E, of the mesylate salt of Compound I. Forms C and D were discovered during the formulation of pharmaceutical compositions with high concentrations of the mesylate salt of Compound I and provided surprisingly high flowability. This enabled accurate dosing of the composition, especially during toxicological studies where compositions with a high concentration is needed so that a high dosage of the compound can be administered to the subject without administering a large volume of the composition. However, Form E did not provide for high flowability at high concentration.

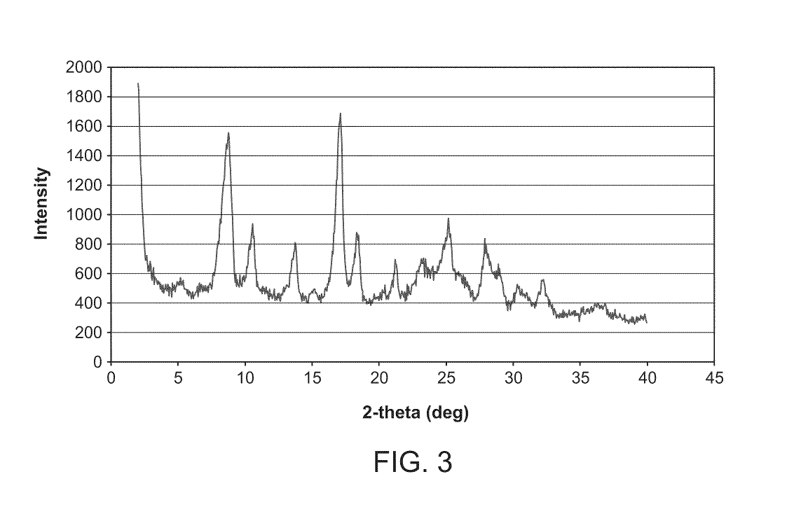

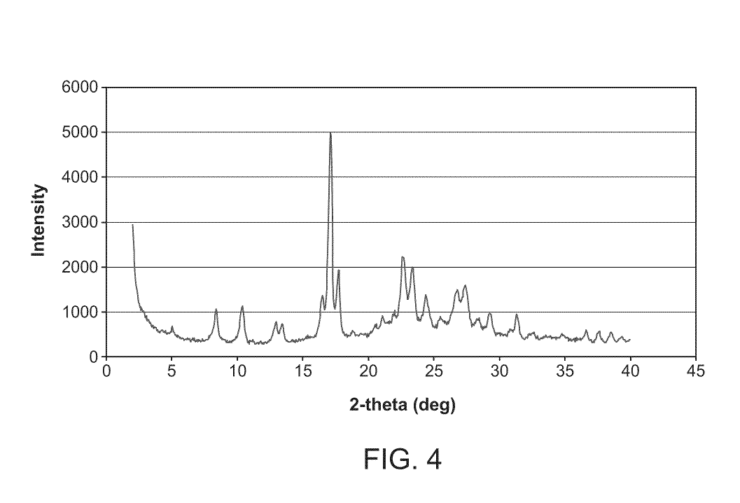

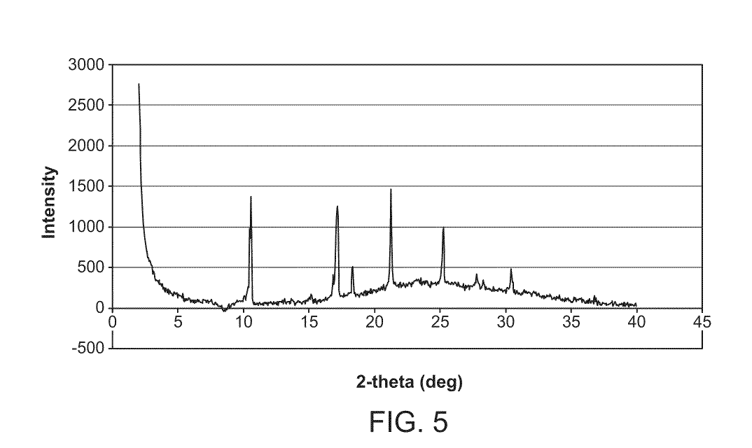

Form C of the mesylate salt of Compound I is characterized by the XRPD shown in FIG. 3. Form D of the mesylate salt of Compound I is characterized by the X-ray diffraction pattern shown in FIG. 4. Form E of the mesylate salt of Compound I is characterized by the X-ray diffraction pattern shown in FIG. 5.

In some embodiments, the crystalline form of Compound I mesylate salt is Form C which shows an XRPD pattern having at least four, six, or eight or all 2θ° peaks selected from the group consisting of about 8.8, about 10.6, about 13.8, about 17.1, about 18.5, about 21.3, about 23.5, about 25.2, about 27.9 and about 29.0. In some embodiments, the crystalline form of Compound I mesylate salt is Form C which shows an XRPD pattern substantially the same as the XRPD pattern of FIG. 3.

In some embodiments, the crystalline form of Compound I mesylate salt is Form D, which shows an XRPD pattern having at least four, six, or eight or all 2θ° peaks selected from the group consisting of about 8.5, about 10.4, about 16.6, about 17.1, about 17.8, about 22.7, about 23.5, about 24.5, about 26.9 and about 27.5. In some embodiments, the crystalline form of Compound I mesylate salt is Form D which shows an XRPD pattern substantially the same as the XRPD pattern of FIG. 4.

In some embodiments, the crystalline form of Compound I mesylate salt is Form E, which shows an XRPD pattern having at least four, six, or eight or all 2θ° peaks selected from the group consisting of about 10.5, about 10.6, about 15.2, about 16.9, about 17.2, about 18.4, about 21.3, about 25.02, about 27.8, about 28.3, and about 30.4. In some embodiments, the crystalline form of Compound I mesylate salt is Form D which shows an XRPD pattern substantially the same as the XRPD pattern of FIG. 5.

The XRPD data provided herein is instrument dependent. The 2θ° peaks provided herein may vary within ±0.2 2θ°, ±0.1 2θ° or ±0.02 2θ°. When two XRDP patterns have at least 4, preferably at least 6, 8, or 10 2θ° peaks, that do not vary more than ±5%, preferably ±1%, more preferably ±0.2% in position and optionally in intensity, it is deemed that the XRDP patterns are substantially the same. Preferably the 4, 6, 8, or 10 peaks are the peaks that have the highest intensity.

In some embodiments, there is provided a mesylate salt of the Compound I wherein at least a portion of the mesylate salt is in a crystalline Form C, D or E. In some embodiments, about or greater than 50% by weight of the mesylate salt of Compound I is present as the polymorphic Form C, D or E. In some embodiments, about or greater than 60% by weight; about or greater than 65% by weight; about or greater than 70% by weight; about or greater than 75% by weight; about or greater than 80% by weight; about or greater than 85% by weight; about or greater than 90% by weight; about or greater than 95% by weight; or about or greater than 99% by weight of the mesylate salt of Compound I is present in the composition as the crystalline Form C, D or E.

The following table provides up to 10 most intense peaks of crystalline forms C, D, and E of the mesylate salt and the free base.

| Form | C | D | E | Free base |
|---|---|---|---|---|
| Angle 2-theta | | | | 5.9 |
| | | | | 6.5 |
| | | 8.5 | | |
| | 8.8 | | | |
| | | 10.4 | | |
| | | | 10.5 | |
| | 10.6 | | 10.6 | |
| | | | | 13.4 |
| | 13.8 | | | |
| | | | | 14.4 |
| | | | | 14.9 |
| | | | 15.2 | |
| | | 16.6 | | |
| | | | 16.9 | |
| | 17.1 | 17.1 | | |
| | | | 17.2 | |
| | | 17.8 | | |
| | | | 18.4 | |
| | 18.5 | | | |
| | | | | 19.1 |
| | | | | 19.7 |
| | 21.3 | | 21.3 | |
| | | 22.7 | | |
| | 23.5 | 23.5 | | |
| | | | | 23.9 |
| | | 24.5 | | |
| | 25.2 | | | 25.2 |
| | | 26.9 | | |
| | | 27.5 | | |
| | | | 27.8 | |
| | 27.9 | | | |
| | | | 28.3 | |
| | 29.0 | | | 29.0 |
| | | | 30.4 | |

The identity of the crystalline forms the present invention can be confirmed by nuclear magnetic resonance (NMR), Fourier transform infrared (FTIR) and mass spectrometry (MS). Purity and water content can be determined by reverse phase high-performance liquid chromatography (HPLC) and Karl Fischer titration method, respectively. Residue solvent content can be determined by gas chromatography (GC). The following are certain analytical methods that can be employed to determine the identity, purity and properties of the crystalline forms of this invention. Exemplary procedures for these analytical methods are described in Example 11 below.
Proton NMR,
FTIR,
Mass Spectroscopy,
HPLC for mesylate content,
Purity was determined based on related substances,
Karl Fischer for water content,
Trace metals and silicon analyses (inductively coupled plasma (ICP) Method),
Elemental analysis by combustion for carbon, hydrogen, nitrogen, by colorimetric titration for sulfur, and by ion chromatography for chlorine
GC for residual solvents.

The crystalline forms provided herein may be obtained by direct crystallization of the salt of Compound I or by crystallization of the salt of Compound I followed by interconversion from another crystalline form or from an amorphous state. Exemplifying procedures are provided in the Examples.

### Therapeutic Methods

The crystalline forms of the mesylate salt of Compound I are useful for preventing or treating a condition in a mammal characterized by undesired thrombosis. In some embodiments, a therapeutically effective amount the crystalline forms of the mesylate salt of Compound I are administered to the mammal in need of such treatment. The crystalline forms of the salt of Compound I can be used either alone or in conjunction with pharmaceutically acceptable excipients to prevent the onset of a condition characterized by undesired thrombosis. Prophylactic treatment can have substantial benefits for a patient at risk of an ailment, through decreased medical treatments and their associated mental and physical costs, as well as the direct monetary savings from avoiding prolonged treatment of a patient. For patients where the condition is not detected sufficiently early to prevent onset, the compounds or salts that can be prepared by the present invention, for example the crystalline forms of the salt of Compound I can be used either alone or in conjunction with pharmaceutically acceptable excipients to treat the condition.

Compound I is characterized by its ability to inhibit thrombus formation with acceptable effects on classical measures of coagulation parameters, platelets and platelet function, and acceptable levels of bleeding complications associated with their use. Conditions characterized by undesired thrombosis would include those involving the arterial and venous vasculature.

With respect to the coronary arterial vasculature, abnormal thrombus formation characterizes the rupture of an established atherosclerotic plaque which is the major cause of acute myocardial infarction and unstable angina, as well as also characterizing the occlusive coronary thrombus formation resulting from either thrombolytic therapy or percutaneous transluminal coronary angioplasty (PTCA).

With respect to the venous vasculature, abnormal thrombus formation characterizes the condition observed in patients undergoing major surgery in the lower extremities or the abdominal area who often suffer from thrombus formation in the venous vasculature resulting in reduced blood flow to the affected extremity and a predisposition to pulmonary embolism. Abnormal thrombus formation further characterizes disseminated intravascular coagulopathy commonly occurs within both vascular systems during septic shock, certain viral infections and cancer, a condition wherein there is rapid consumption of coagulation factors and systemic coagulation which results in the formation of life-threatening thrombi occurring throughout the microvasculature leading to widespread organ failure.

The crystalline forms of the mesylate salt of Compound I of the present invention, selected and used as disclosed herein, are useful for preventing or treating a condition characterized by undesired thrombosis, such as (a) the treatment or prevention of any thrombotically mediated acute coronary syndrome including myocardial infarction, unstable angina, refractory angina, occlusive coronary thrombus occurring post-thrombolytic therapy or post-coronary angioplasty, (b) the treatment or prevention of any thrombotically mediated cerebrovascular syndrome including embolic stroke, thrombotic stroke or transient ischemic attacks, (c) the treatment or prevention of any thrombotic syndrome occurring in the venous system including deep venous thrombosis or pulmonary embolus occurring either spontaneously or in the setting of malignancy, surgery or trauma, (d) the treatment or prevention of any coagulopathy including disseminated intravascular coagulation (including the setting of septic shock or other infection, surgery, pregnancy, trauma or malignancy and whether associated with multi-organ failure or not), thrombotic thrombocytopenic purpura, thromboangiitis obliterans, or thrombotic disease associated with heparin induced thrombocytopenia, (e) the treatment or prevention of thrombotic complications associated with extracorporeal circulation (e.g. renal dialysis, cardiopulmonary bypass or other oxygenation procedure, plasmapheresis), (f) the treatment or prevention of thrombotic complications associated with instrumentation (e.g. cardiac or other intravascular catheterization, intra-aortic balloon pump, coronary stent or cardiac valve), and (g) those involved with the fitting of prosthetic devices.

In some embodiments, the crystalline forms of the mesylate salt of Compound I are useful in treating thrombosis and conditions associated with thrombosis. Accordingly, a method for preventing or treating a condition in a mammal characterized by undesired thrombosis comprises administering to the mammal a therapeutically effective amount of a salt of this invention. The crystalline forms of Compound I are useful in treating undesired thrombosis and/or associated conditions including, but not limited to, acute coronary syndrome, myocardial infarction, unstable angina, refractory angina, occlusive coronary thrombus occurring post-thrombolytic therapy or post-coronary angioplasty, a thrombotically mediated cerebrovascular syndrome, embolic stroke, thrombotic stroke, transient ischemic attacks, venous thrombosis, deep venous thrombosis, pulmonary embolus, coagulopathy, disseminated intravascular coagulation, thrombotic thrombocytopenic purpura, thromboanglitis obliterans, thrombotic disease associated with heparin-induced thrombocytopenia, thrombotic complications associated with extracorporeal circulation, thrombotic complications associated with instrumentation, thrombotic complications associated with the fitting of prosthetic devices, occlusive coronary thrombus formation resulting from either thrombolytic therapy or percutaneous transluminal coronary angioplasty, thrombus formation in the venous vasculature, disseminated intravascular coagulopathy, a condition wherein there is rapid consumption of coagulation factors and systemic coagulation which results in the formation of life-threatening thrombi occurring throughout the microvasculature leading to widespread organ failure, hemorrhagic stroke, renal dialysis, blood oxygenation, and cardiac catheterization.

In some embodiments, the condition is selected from the group consisting of embolic stroke, thrombotic stroke, venous thrombosis, deep venous thrombosis, acute coronary syndrome, and myocardial infarction.

In some embodiments, the crystalline forms of the mesylate salt of Compound I are useful in: prevention of stroke in atrial fibrillation patients; prevention of thrombosis in medically ill patients; prevention and treatment of deep vein thrombosis; prevention of arterial thrombosis in acute coronary syndrome patients; and/or secondary prevention of myocardial infarction, stroke or other thrombotic events in patients who have had a prior event.

The crystalline form of the mesylate salt of Compound I of this invention can also be used whenever inhibition of blood coagulation is required such as to prevent coagulation of stored whole blood and to prevent coagulation in other biological samples for testing or storage. Thus coagulation inhibitors of the present inhibition can be added to or contacted with stored whole blood and any medium containing or suspected of containing plasma coagulation factors and in which it is desired that blood coagulation be inhibited, e.g. when contacting the mammal's blood with material selected from the group consisting of vascular grafts, stents, orthopedic prosthesis, cardiac prosthesis, and extracorporeal circulation systems.

Besides being useful for human treatment, the crystalline forms are also useful for veterinary treatment of companion animals, exotic animals and farm animals, including mammals, rodents, and the like. More preferred animals include horses, dogs, and cats.

### Pharmaceutical Compositions and Administration

The pharmaceutical compositions comprising the crystalline forms of the mesylate salt of Compound I described herein can be used for preventing or treating a subject suffering from a disease condition, wherein the disease condition is characterized by undesired thrombosis. In some embodiments, the pharmaceutical compositions are comprised of a pharmaceutically acceptable carrier and a therapeutically effective amount of the crystalline polymorph forms of the mesylate Compound I.

In the management of thrombotic disorders, the crystalline forms of the mesylate salt of Compound I may be utilized in compositions such as tablets, capsules, lozenges or elixirs for oral administration, suppositories, sterile solutions or suspensions or injectable administration, and the like, or incorporated into shaped articles. Subjects in need of treatment (typically mammalian) using the crystalline forms of the mesylate salt of Compound I of this invention can be administered in dosages that will provide optimal efficacy. The dose and method of administration will vary from subject to subject and be dependent upon such factors as the type of mammal being treated, its sex, weight, diet, concurrent medication, overall clinical condition, the particular compounds employed, the specific use for which these compounds are employed, and other factors which those skilled in the medical arts will recognize

Typical adjuvants which may be incorporated into tablets, capsules, lozenges and the like are binders such as acacia, corn starch or gelatin, and excipients such as microcrystalline cellulose, disintegrating agents like corn starch or alginic acid, lubricants such as magnesium stearate, sweetening agents such as sucrose or lactose, or flavoring agents. Sterile compositions for injection can be formulated according to conventional pharmaceutical practice.

Capsules useful in the present invention can be prepared using conventional and known encapsulation techniques, such as that described in Stroud et al., U.S. Pat. No. 5,735,105. The capsule is typically a hollow shell of generally cylindrical shape having a diameter and length sufficient so that the pharmaceutical compositions containing the appropriate dose of the active agent fit inside the capsule. The interior of the capsules can include plasticizer, gelatin, modified starches, gums, carrageenans and mixtures thereof. Liquid carriers such as water, saline, or a fatty oil can also be present. Those skilled in the art will appreciate what compositions are suitable.

In addition to the active agent, tablets useful in the present invention can comprise fillers, binders, compression agents, lubricants, disintegrants, colorants, water, talc and other elements recognized by one of skill in the art. The tablets can be homogeneous with a single layer at the core, or have multiple layers in order to realize preferred release profiles. In some instances, the tablets of the instant invention may be coated, such as with an enteric coating. One of skill in the art will appreciate that other excipients are useful in the tablets of the present invention.

Lozenges useful in the present invention include an appropriate amount of the active agent as well as any fillers, binders, disintegrants, solvents, solubilizing agents, sweeteners, coloring agents and any other ingredients that one of skill in the art would appreciate is necessary. Lozenges of the present invention are designed to dissolve and release the active agent on contact with the mouth of the patient. One of skill in the art will appreciate that other delivery methods are useful in the present invention.

Formulations of the crystalline forms of the mesylate salt of this invention are prepared for storage or administration by mixing the compound having a desired degree of purity with physiologically acceptable carriers, excipients, stabilizers etc., and may be provided in sustained release or timed release formulations. Pharmaceutically acceptable carriers or diluents for therapeutic use are well known in the pharmaceutical field, and are described, for example, in Remington's Pharmaceutical Sciences, Mack Publishing Co., (A. R. Gennaro edit. 1985). Such materials are nontoxic to the recipients at the dosages and concentrations employed, and include buffers such as phosphate, citrate, acetate and other organic acid salts, antioxidants such as ascorbic acid, low molecular weight (less than about ten residues) peptides such as polyarginine, proteins, such as serum albumin, gelatin, or immunoglobulins, hydrophilic polymers such as polyvinylpyrrolidinone, amino acids such as glycine, glutamic acid, aspartic acid, or arginine, monosaccharides, disaccharides, and other carbohydrates including cellulose or its derivatives, glucose, mannose or dextrins, chelating agents such as EDTA, sugar alcohols such as mannitol or sorbitol, counterions such as sodium and/or nonionic surfactants such as Tween, Pluronics or polyethyleneglycol.

The term “pharmaceutically acceptable aqueous carrier” refers to water or a pharmaceutically acceptable aqueous solution of a pharmaceutically acceptable excipient in water, including aqueous solutions of protamine sulfate, disodium hydrogen phosphate, potassium hydrogen phosphate, sodium chloride, zinc salts, magnesium trisilicate, polyvinyl pyrrolidone, cellulose-based substances, polyethylene glycol, sodium carboxymethylcellulose, polyacrylates, glucose, mannose or dextrins, chelating agents such as EDTA, sugar alcohols such as mannitol or sorbitol, etc. such as saline, or phosphate buffer.

Dosage formulations of the crystalline forms of the mesylate salt of this invention to be used for therapeutic administration may be sterile. Sterility is readily accomplished by filtration through sterile membranes such as 0.2 micron membranes, or by other conventional methods. Formulations typically will be stored in lyophilized form or as an aqueous solution. The pH of the preparations of this invention typically will be between 3 and 11, more preferably from 5 to 9 and most preferably from 7 to 8. It will be understood that use of certain of the foregoing excipients, carriers, or stabilizers will result in the formation of cyclic polypeptide salts. Route of administration may by injection, such as intravenously (bolus and/or infusion), subcutaneously, intramuscularly, colonically, rectally, nasally or intraperitoneally, or employing a variety of dosage forms such as suppositories, implanted pellets or small cylinders, aerosols, oral dosage formulations (such as tablets, capsules and lozenges) and topical formulations such as ointments, drops and dermal patches. The sterile of this invention are desirably incorporated into shaped articles such as implants which may employ inert materials such as biodegradable polymers or synthetic silicones, for example, Silastic, silicone rubber or other polymers commercially available.

The crystalline forms of the invention may also be administered in the form of liposome delivery systems, such as small unilamellar vesicles, large unilamellar vesicles and multilamellar vesicles. Liposomes can be formed from a variety of lipids, such as cholesterol, stearylamine or phosphatidylcholines.

The crystalline forms of the mesylate salt of this invention may also be delivered by the use of antibodies, antibody fragments, growth factors, hormones, or other targeting moieties, to which the salt molecules are coupled. The crystalline forms of this invention may also be coupled with suitable polymers as targetable drug carriers. Such polymers can include polyvinylpyrrolidinone, pyran copolymer, polyhydroxy-propyl-methacrylamide-phenol, polyhydroxyethyl-aspartamide-phenol, or polyethyleneoxide-polylysine substituted with palmitoyl residues. Furthermore, the crystalline forms of the invention may be coupled to a class of biodegradable polymers useful in achieving controlled release of a drug, for example polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels. Polymers and semipermeable polymer matrices may be formed into shaped articles, such as valves, stents, tubing, prostheses and the like.

In some embodiments, the pharmaceutical composition comprises a pharmaceutically acceptable carrier and the crystalline forms of the mesylate salt of Compound I, wherein the pharmaceutical composition is in a solid form or a suspension in a liquid excipient and the crystalline form of the mesylate salt of Compound I provides improved thermo and/or hydrolytic stability, handling, purity, which provides improved efficacy and/or safety profile.

In some embodiments, the pharmaceutical composition comprises a pharmaceutically acceptable carrier and is prepared from the crystalline form of the salt of Compound I, wherein the pharmaceutical composition is in a liquid solution form and the crystalline form of Compound I provides improved thermo and hydrolytic stability, handling, purity and solubility, which provides improved efficacy and/or safety profile. Liquid formulations of the crystalline forms of Compound I can be prepared by, for example, dissolution or suspension of the active compound in a vehicle such as an oil or a synthetic fatty vehicle like ethyl oleate, or into a liposome may be desired. Buffers, preservatives, antioxidants and the like can be incorporated according to accepted pharmaceutical practice.

Therapeutic compound liquid formulations generally are placed into a container having a sterile access port, for example, an intravenous solution bag or vial having a stopper pierceable by hypodermic injection needle.

Typically, about 0.5 to 500 mg of the crystalline form of the salt of Compound I is compounded with a physiologically acceptable vehicle, carrier, excipient, binder, preservative, stabilizer, dye, flavor etc., as called for by accepted pharmaceutical practice. The amount of active ingredient in these compositions is such that a suitable dosage in the range indicated is obtained.

It is contemplated that a typical dosage of Compound I will range from about 0.001 mg/kg to about 1000 mg/kg, preferably from about 0.01 mg/kg to about 100 mg/kg, and more preferably from about 0.10 mg/kg to about 20 mg/kg. Advantageously, the crystalline forms of this invention may be administered several times daily, and other dosage regimens may also be useful.

Therapeutically effective dosages may be determined by either in vitro or in vivo methods. The range of therapeutically effective dosages will be influenced by the route of administration, the therapeutic objectives and the condition of the patient. For injection by hypodermic needle, it may be assumed the dosage is delivered into the body's fluids. For other routes of administration, the absorption efficiency may need to be individually determined for by methods well known in pharmacology. Accordingly, it may be necessary for the therapist to titer the dosage and modify the route of administration as required to obtain the optimal therapeutic effect. The determination of effective dosage levels, that is, the dosage levels necessary to achieve the desired result, will be readily determined by one skilled in the art. Typically, applications of Compound I are commenced at lower dosage levels, with dosage levels being increased until the desired effect is achieved.

During toxicology studies, it has been discovered that crystalline Forms A has limited flowability (i.e., difficult to gavage) at concentrations at or above 10-30 mg/mL, mesylate Form B has limited flowability at concentrations at or above 1 mg/mL. Aqueous suspension of Form E is not free-flowing. It was surprisingly discovered that crystalline Form C and Form D, provide free-flowing aqueous suspensions at concentrations of at least as high as 100 mg/mL of Compound I. This allows toxicology studies to dose up to at least 2 g/kg.

Thus, in another aspect, this invention provides a composition which is a free-flowing dosable aqueous suspension, wherein the composition comprises a pharmaceutically acceptable aqueous carrier and a mesylate salt of the compound of 5-chloro-N-((1-(4-(2-oxopyridin-1(2H)-yl)phenyl)-1H-imidazol-4-yl)methyl)thiophene-2-carboxamide or a tautomer thereof; wherein the concentration of the compound is at least 40 mg/mL, and wherein at least a portion of the salt is in a crystalline form characterized by an X-ray powder diffraction (XRPD) pattern substantially the same as:

(a) an X-ray powder diffraction (XRPD) pattern having at least six, or eight or ten 2θ° peaks selected from the group consisting of 8.8, 10.6, 13.8, 17.1, 18.5, 21.3, 23.5, 25.2, 27.9 and 29.0 or substantially the XRPD as provided in FIG. 3; or
(b) an X-ray powder diffraction (XRPD) pattern having at least six or eight or ten 2θ° peaks selected from the group consisting of 8.5, 10.4, 16.6, 17.1, 17.8, 22.7, 23.5, 24.5, 26.9 and 27.5 or substantially the XRPD as provided in FIG. 4;
or in an amorphous form,
or combinations thereof.

In still another aspect, this invention provides a method of preparing the above pharmaceutical composition, which method comprises mixing the mesylate salt of 5-chloro-N-((1-(4-(2-oxopyridin-1(2H)-yl)phenyl)-1H-imidazol-4-yl)methyl)thiophene-2-carboxamide or tautomer thereof and the pharmaceutically acceptable aqueous carrier, and storing the mixture at a temperature of about or below 8° C. for a period of time sufficient to form a free-flowing composition. In some embodiments, the method comprises mixing Form A or Form B of the mesylate salt of 5-chloro-N-((1-(4-(2-oxopyridin-1(2H)-yl)phenyl)-1H-imidazol-4-yl)methyl)thiophene-2-carboxamide or tautomer thereof and the pharmaceutically acceptable aqueous carrier, and storing the mixture at a temperature of about or below 8° C. for a period of time sufficient to allow the Form A or Form B to covert to Form C or Form D thereby forming a free-flowing composition. Preferably, the mixing is sufficient to help the mixing/wetting of Form A or Form B of the mesylate salt of Compound I with the aqueous pharmaceutical carrier. Effective mixing can be achieved by using a planetary mixer, such as a mixer by THINKY, Corp., a homogenizer or ball milling. The storage temperature under which conversion from Form A or B to Form C or D occurs is generally at or below refrigerated temperatures, such as at or below about 2-8° C. or at or below about 4° C.

The amorphous form of the mesylate salt of Compound I may be prepared by deep freezing the salt at about −70° C. to about −80° C. (deep freezer).

### EXAMPLES

Unless stated otherwise, the abbreviations used throughout the specification have the following meanings:
A %=total percent area
aq.=aqueous
AUC=area under curve
CH₃SO₃H=methanesulfonic acid
cm=centimeter
CuI=copper (I) iodide
d=doublet
deg=degree
DIPEA=diisopropylethylamine
DMSO=dimethyl sulfoxide
DSC=differential scanning calorimetry
EDTA=ethylenediaminetetraacetic acid
eq. or equiv=equivalent
Et₃SiH=triethyl silane
EtOAc=ethyl acetate
EtOH=ethanol
g=gram
H₂SO₄=sulfuric acid
HPLC=high performance liquid chromatography
hr=hour
Hz=hertz
FTIR=Fourier transform infrared
IC=ion chromatography
ICP=inductively coupled plasma
IPA=Isopropanol
IR=infrared
J=coupling constant
K₂CO₃=potassium carbonate
kg=kilogram
L=liter
LOD=limit of detection
M=molar
m=multiplet
mA=milliampere
Me=methyl
MeCN=acetonitrile
MEK=methyl ethyl ketone
MeO=methoxy
MeOH=methanol
MeTHF=methyltetrahydrofuran
mg=milligram
min.=minute
mL=milliliter
mm=millimeter
mmHg=millimeters of mercury
MTBE=methyl tert butyl ether
N=normal
Na₂SO₄=sodium sulfate
NH₃=ammonia
nM=nanomolar
NMR=nuclear magnetic resonance
PhMe=toluene
ppm=parts per million
RH=relative humidity
rpm=revolutions per minute
r.t.=room temperature
s=singlet
TGA=thermal gravimetric analysis
TDS=total dissolved solids
TFA=trifluoroacetic acid
THF=tetrahydrofuran
Wt=weight
μM=micromolar

U.S. Pat. No. 7,763,608 and U.S. Provisional Patent Application No. 61/287,679, filed Dec. 17, 2009, and U.S. patent application Ser. No. 12/______, Attorney docket no. 070545-1851, filed on Dec. 16, 2010, both titled “Methods Of Preparing Factor Xa Inhibitors And Salts Thereof,” and U.S. Pat. No. 7,763,608, describe processes of preparing Compound I, all of which are incorporated by reference in their entirety.

### Example 1

### Preparation of Compound C

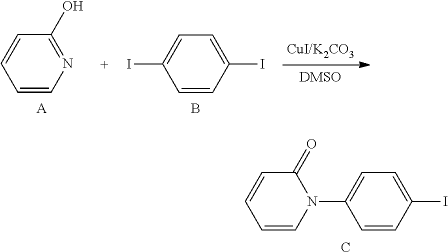

121.1 kg of 1,4-diiodobenzene B (0.367 mol) was charged as a solid to a 200-gallon reactor containing DMSO followed by 35 kg of 2-hydroxypyridine A (0.368 mol), 123 kg of K₂CO₃ (4.8 equiv), and 7.3 kg CuI (0.1 equiv). The mixture was heated to 120±5° C. for 3 hours. The reaction monitored by HPLC for completion. The reaction after cooling was quenched with water and ethyl acetate (EtOAc). The organic layer was washed with brine followed by Na₂SO₄ drying. After filtration, the EtOAc layer was concentrated at 85° C. followed by addition of heptanes. The slurry was then cooled to 20° C. for 1 hour and isolated via a cleaned and dried centrifuge. The product was collected and dried for 16 hours at 35° C. under about 28 mmHg. Recovery was 55.1 kg (50% yield) of Compound C, with 85.9% AUC purity.

### Example 2

### Preparation of Compound E

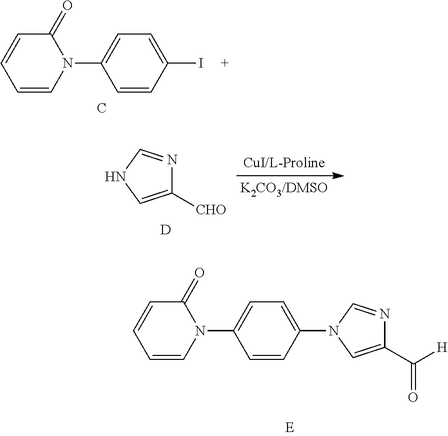

CuI (2.95 kg, 0.20 equiv) and L-proline (1.88 kg, 0.20 equiv) were mixed for 15 minutes in DMSO (253 kg). To this was added Compound C (24 kg, 1.0 equiv), K₂CO₃ (22.8 kg, 4.1 equiv), and 4-formylimidazole D (8.30 kg, 1.07 equiv). The reaction mixture was then heated to 120±5° C. for 3.5 hours. HPLC analysis concluded the reaction was complete and the mixture was cooled to 20±5° C. The reaction was diluted with water (40 volumes) and dichloromethane (DCM) (20 volumes), stirred for 1 hour, and centrifuged. In order to remove the insoluble impurities, the centrifuge filtrates were then polished filtered across a press fitted with 1-micron paper. The layers were separated, organic layer dried with Na₂SO₄, and filtered. The organic filtrates were atmospherically distilled to reduce the volume, charged with EtOAc, and continued to distill to an internal temperature of about 72° C. The product slurry was cooled to about 20° C. for 2 hours and isolated 6.5 kg of the aldehyde intermediate Compound E (30.4% yield).

### Example 3

### Preparation of Compound G

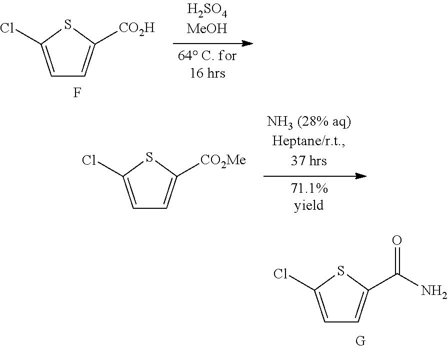

The 200-gallon reactor was charged with methanol (135 kg), 5-chlorothiophene-2-carboxylic acid F (12 kg, 73.81 mol, 1.0 equiv), and sulfuric acid (6.7 kg, 68.31 mol) under nitrogen. The contents were warmed to reflux (64° C.) for 16 hours and reaction completion monitored by HPLC. The reactor was cooled to 40° C. and the mixture was vacuum distilled to an oil at <50° C. The methyl ester obtained was cooled to 20° C. and ammonium hydroxide (157 kg, 2586 mol, 35.2 equiv) was charged along with heptane (10 kg) to the reactor. The reactor contents were mixed for 36 hours at ambient temperature. The completion of the reaction was monitored by HPLC for the disappearance of methyl ester intermediate. The precipitated solids were centrifuged and washed with water followed by heptane. The isolated solids were dried at 45° C. for 14 hours to afford Compound G (8.42 kg, 77.1% yield).

### Example 4

### Preparation of Compound I

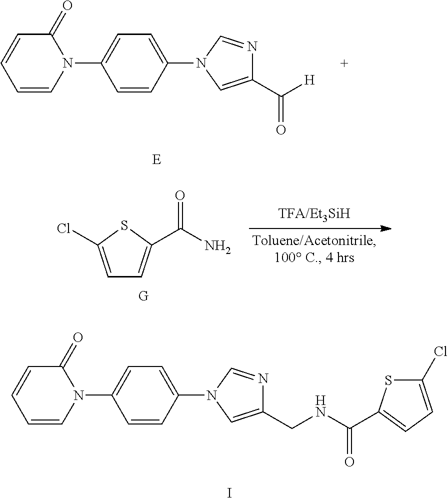

A nitrogen-inerted 200-gallon reactor was charged with toluene (202.4 kg), 1-(4-(2-oxopyridin-1(2H)-yl)phenyl)-1H-imidazole-4-carbaldehyde (Compound E, 11.7 kg, 1.0 equiv) and 2-amido-5-chlorothiophene (Compound G, 7.8 kg, 1.09 equiv). The contents were mixed together for 15 minutes followed by the addition of triethylsilane (Et₃SiH) (15.3 kg, 3.0 equiv) and trifluoroacetic acid (15.3 kg, 3.0 equiv). The reaction mixture was heated at 100±5° C. for 4 hours and the reaction monitored by HPLC analysis. The reaction was complete when Compound E was less than 1%. The reaction mixture was cooled to 40±5° C. and acetonitrile (139.2 kg) was added and reaction further cooled to 15±5° C. While maintaining the temperature at or below about 35° C., diisopropylethylamine (DIPEA) (18.7 kg, 3.3 equiv) was added over 15 minutes. The reaction contents were stirred for 1 hour at 20° C. and the solid Compound I isolated on a centrifuge. The collected free base of Compound I was vacuum dried at 45° C. and 28 mmHg for 32 hours. Compound I (10.89 kg, 60.2% yield) was collected as tan solid. HPLC record purity as area %: 93.5%. Structure was confirmed by Infrared (IR) spectrum.

### Example 5

### Preparation of Methanesulfonate (Mesylate) Salt of Compound I

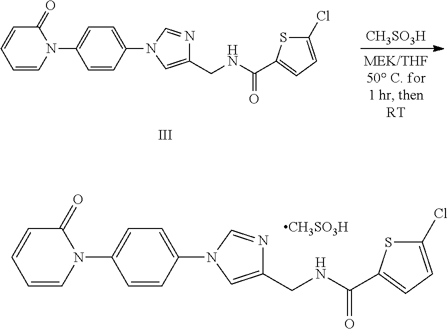

A slurry of free base of Compound I (10.89 kg, 1.0 equiv) in methylethyl ketone (MEK, 217 kg) was mixed for 30 minutes. To this slurry a THF solution (27.2 kg, 2.8 volumes) of methanesulfonic acid (MeSO₃H) (2.72 kg, 1.07 equiv) was added and the mixture heated at 50° C. for 1 hour. The mixture was cooled to 20° C. and stirred for 30 minutes. The mesylate salt was centrifuged and washed with MEK (32.7 kg). The product was dried at 45° C. under 28 mmHg vacuum for 116 hours and further dried at 61° C. for 60 hours. After drying, 12.50 kg the mesylate salt of Compound I (95% yield) was isolated in crystalline Form A (XRPD shown in FIG. 1).

### Example 6

### Preparation of Methanesulfonate (Mesylate) Salt of Compound I

The mesylate salt of Compound 12.70 kg was charged to a glass-lined reactor, followed by acetone (39.70 kg), and USP water (4.35 kg). The solution was refluxed at 58° C. for approximately 1 hour followed by hot polish filtration through 0.2-micron cartridge filter. The polished filtrate was cooled to 20±5° C. followed by addition of methylethylketone (MEK) (32.80 kg) and stirred for 12 hours at ambient temperature. The slurry was cooled to 0-5° C. for more than 2 hours and then filtered through a filtration funnel. The solid isolated was dried at 50±5° C. under vacuum for at least 16 hours to afford the mesylate salt of Compound I (1.7 kg) as a tan crystalline Form B (XRPD shown in FIG. 2).

### Example 7

### Preparation of Form C of Compound I Mesylate Salt

A formulation comprises of crystalline Form A of Compound I mesylate salt up to 100 mg/mL suspended in an aqueous vehicle of 0.3% methylcellulose, 0.5% polysorbate 80, and 0.25% simethicone. Since Compound I is difficult to wet and disperse, especially at high doses, a planetary mixer (e.g., a mixer by THINKY Corp.) was used. At 100 mg/mL Compound I, the suspension was off white in color, very thick and flow poorly immediately after mixing. However, after brief storage (<1 hour) at refrigerated condition, the suspension became free-flowing. It was found that the Form A of Compound I changed to a different white crystalline Form C (XRPD shown in FIG. 3). It has been found that effective mixing of Form A of Compound I under high shear, such as by a THINKY mixer or using a homogenizer or ball milling facilitates fast and complete conversion of Form A to Form C. Crystalline Form C is both physically and chemically stable at refrigerated condition for at least one week.

### Example 8

### Preparation of Form D of Compound I Mesylate Salt

In another experiment, a formulation of 100 mg/mL Form A of Compound I mesylate salt did not become free-flowing, i.e., did not convert to Form C after storage at refrigerated condition. In order to obtain a dosable formulation, the procedure was revised to include free-flowing crystalline form and multiple freezing (at −20° C.)-thawing-mixing cycles to speed up the conversion. After this process, a free-flowing crystalline form was obtained having a unique XRPD pattern and was named Form D (XRPD shown in FIG. 4).

### Example 9

### Preparation of an Amorphous Form of Compound I Mesylate Salt

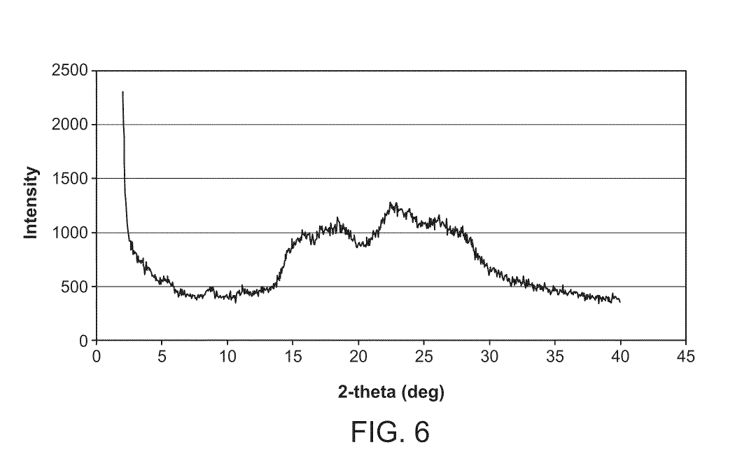

It was found that during the formulation of crystalline Form A, the suspension became gelled after mixing and Form A converted to an amorphous form after mixing with vehicle (XRPD shown in FIG. 6). This formulation gelled at room temperature and did not flow. Storage at refrigerated condition did not improve the flow. The previous multiple freezing-thawing-mixing cycles developed are tedious and not practical in larger scale. In order to obtain a flowable formulation for dosing, a deep freezing (−80° C.) and thawing cycle was developed to minimize the gelling and convert the formulation to a dark brown, flowing and dosable suspension. The suspended particles maintained its amorphous identity and did not convert to the free-flowing white crystalline form at least in a month at room temperature or refrigerator. The new formulation was found to be also chemically stable at refrigerated condition for at least 1 week.

### Example 10

### Preparation of Form E of Compound I Mesylate Salt

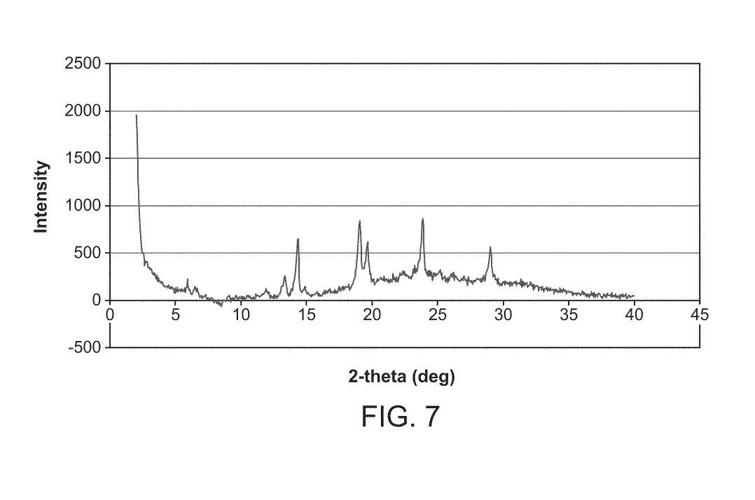

The crystalline mesylate Form B after dispersing in water, overnight at room temperature converted partially to a needle shaped crystalline Form E with a distinctive XRPD pattern (FIG. 5). In addition, similar to the formulation described above, the suspension gelled and lost flowability. In order to obtain a free-flowing dispersion with better controlled drug particle size and solid state, warm (control temperature range) phosphate buffer was added into warm (control temperature range) Compound I aqueous dispersion and Compound I mesylate was converted to free base, which showed the same XRPD pattern of the single base form that has been observed to date (XRPD shown in FIG. 7, prominent 2θ° peaks being 5.9, 6.5, 13.4, 14.4, 14.9, 19.1, 19.7, 23.9, 25.2 and 29.0).

### Example 11

### Analytical Procedures

The following were performed or may be performed to analyze the crystalline forms of the mesylate salt of Compound I of this invention.
Proton NMR,
FTIR spectroscopy,
mass spectroscopy (MS),
HPLC for mesylate content,
Purity was determined based on related substances (HPLC), and
Elemental analysis by combustion for carbon, hydrogen, nitrogen, by colorimetric titration for sulfur, and by ion chromatography for chlorine.

### X-Ray Powder Diffraction (XRPD)

X-Ray Powder Diffraction patterns were collected on a Bruker AXS C2 GADDS diffractometer by Bruker AXS Inc., Madison, Wis., USA or equivalent using Cu Kα radiation (40 kV, 40 mA), automated XYZ stage, laser video microscope for auto-sample positioning and a HiStar 2-dimensional area detector. X-ray optics consists of a single Gael multilayer mirror coupled with a pinhole collimator of 0.3 mm.

The beam divergence, i.e. the effective size of the X-ray beam on the sample, was approximately 4 mm. A θ-θ continuous scan mode was employed with a sample-detector distance of 20 cm which gives an effective 2θ range of 3.2°-29.7°. Typically the sample would be exposed to the X-ray beam for 120 seconds.

### Differential Scanning Calorimetry (DSC)

DSC data can be collected on a Mettler DSC 823e by Mettler-Toledo Inc., Columbus, Ohio, USA equipped with a 50 position auto-sampler. The instrument can be calibrated for energy and temperature using certified indium. Typically 0.5-3 mg of each sample, in a pin-holed aluminum pan, is heated at 10° C./min from 25° C. to 350° C. A nitrogen purge at 50 mL/min is maintained over the sample. The instrument control and data analysis software can be STARe v9.01.

### Thermodynamic Aqueous Solubility by HPLC (Filtration Method)

Aqueous solubility can be determined by suspending sufficient compound in water to give a maximum final concentration of >10 mg/mL of the parent free-form of the compound. The suspension is equilibrated at 25° C. for 24 hours then the pH was measured. The suspension is then filtered through a glass fiber C filter into a 96 well plate. The filtrate is then diluted by a factor of 101. Quantitation can be done by HPLC with reference to a standard solution of approximately 0.1 mg/mL in DMSO. Different volumes of the standard, diluted and undiluted sample solutions are injected. The solubility can be calculated using the peak areas determined by integration of the peak found at the same retention time as the principal peak in the standard injection.

### Thermodynamic Aqueous Solubility by HPLC (Ultracentrifugation Method)

Aqueous solubility can be determined by suspending sufficient compound in water to give a maximum final concentration of ≧10 mg/mL of the parent free-form of the compound. The mixture is allowed to equilibrate for at least 48 hours at 37° C. under mild agitation. At each 24-48 hour interval, an aliquot is removed and the solution is separated from the solid via ultracentrifugation at >60,000 rpm for 20 min and then analyzed by HPLC for the concentration of the compound in the supernatant. Upon equilibration (concentration plateau is reached), the concentration is reported as solubility. This method is the preferred method in determining solubility as it attains the equilibrium and is measurement of the thermodynamic solubility.

### pKa Determination and Prediction:

Data can be collected on a Sirius GlpKa instrument by Sirius Analytical Ltd., UK with a D-PAS attachment. Measurements can be made at 25° C. in aqueous solution by UV and in methanol water mixtures by potentiometry. The titration media can be ionic-strength adjusted (USA) with 0.15 M KCl (aq). The values found in the methanol water mixtures are corrected to 0% co-solvent via a Yasuda-Shedlovsky extrapolation. The data can be refined using Refinement Pro software v1.0. Prediction of pKa values can be made using ACD pKa prediction software v9.

### Log P & Log D Determination

Data can be collected by potentiometric titration on a Sirius GlpKa instrument using three ratios of octanol: ionic-strength adjusted (USA) water to generate Log P, Log Pion, and Log D values. The data can be refined using Refinement Pro software v1.0. Prediction of Log P values can be made using ACD v9 and KOWWIN™ v1.67 software from Syracuse Research Corp., Syracuse, N.Y., USA.

### Gravimetric Vapour Sorption.

Sorption isotherms can be obtained using a SMS HT-DVS moisture sorption analyzer by Surface Measurement Systems Limited, Middlesex, UK, controlled by SMS Analysis Suite software. The sample temperature is maintained at 25° C. by the instrument controls. The humidity is controlled by mixing streams of dry and wet nitrogen, with a total flow rate of 400 mL/min. The relative humidity is measured by a calibrated optical dew point transmitter (dynamic range of 0.5-100% RH), located near the sample. The weight change, (mass relaxation) of the sample as a function of % RH is constantly monitored by the microbalance (accuracy ±0.005 mg).

Typically 5-20 mg of sample is placed in a tared mesh stainless steel liner within a stainless steel pan under ambient conditions. The sample is loaded and unloaded at 40% RH and 25° C. (typical room conditions).

### Example 12

The following experiments were conducted using samples of Compound I prepared by a method disclosed in U.S. Pat. No. 7,763,608, which is incorporated by reference in its entirety.

Compound I was used in the rat investigation. An intravenous (IV) and oral (PO) dose of Compound I (1.0 and 10 mg/kg, respectively) was prepared. The IV dose was solubilized in 50% PEG300 to yield a final concentration of 1.0 mg/mL with a final pH of 5.13. The PO dose was suspended in 0.5% methylcellulose at a concentration of 2.0 mg/mL with a final pH of 2.70.

For the dog and monkey study, Compound I was also used. An IV and PO dose of Compound I (1.0 and 5.0 mg/kg, respectively) was prepared. The IV dose was formulated similarly to that used in the rat study (50% PEG300 in water). The PO dose was suspended in 0.5% methylcellulose at a concentration of 1.0 mg/mL with a final pH of approximately 3.50.

### Study Design

A total of six male Sprague-Dawley rats (n=3/dosing group) from Charles River Laboratories (Hollister, Calif.), three male beagle dogs from Marshall BioResources (North Rose, N.Y.) and three male rhesus monkeys were utilized. All surgical procedures in rat (femoral and jugular vein catheterizations) were performed 8 days prior to utilization in the study and rats were acclimated in-house 5 days prior to utilization. Dogs were acclimated in-house at least seven days prior to utilization and were returned to the colony at the completion of the study. Monkey studies were conducted by an off-site contract laboratory.

All animals were fasted from the afternoon prior to study initiation to two hours post-dose (approximately 18 hours). Water was provided ad libitum. All animal rooms were on a 12 hour light-dark cycles (6 A.M. to 6 P.M.). On the morning of experimentation, animals were weighed. Rat femoral and jugular (IV only) vein blood lines were exteriorized and attached to access ports. Dogs were weighed and shaved at blood sampling and IV dosing sites (along both cephalic and saphenous veins).

All animals were dosed based on individual weights with a PO gavage volume of 5.0 mL/kg and an IV bolus dose volume of 1.0 mL/kg. Blood samples were obtained on 3.8% TSC (1:10 dilution) over a 24, 56, and 96 hour period post-dosing for the rat, dog, and monkey, respectively. Blood samples were centrifuged for platelet poor plasma, and resulting plasma was stored at −20° C. until sample analysis. Rat urine samples were collected on 200 μL of 2% boric acid from animals in the IV group at 0 (overnight), 10, and 24 hours post-dose. At collection times, urine volume and water consumption was recorded. Urine samples were stored at −20° C. until sample analysis.

### Sample Analysis

Plasma and urine samples were analyzed for Compound I concentration using a liquid chromatography tandem mass spectrometry (LC/MS/MS). In brief, plasma and urine samples were processed in a 96-well Captiva™ filter plate (0.2 μm, Varian, Inc., Palo Alto, Calif.). Aliquots of plasma samples were precipitated with acetonitrile containing 500 ng/mL of N-(2-(5-chloropyridin-2-ylcarbaomoyl)-4-methoxyphenyl)-4-(N,N-dimethylcarbamimidoyl)-2-fluorobenzamide, an internal standard. Aliquots of urine samples were diluted with plasma before mixing with acetonitrile containing internal standard. The mixture was vortexed and refrigerated at 4° C. for 30 minutes to allow complete protein precipitation. The mixture was filtered into a 96-well collection plate. The filtrate was injected onto a Sciex API3000 LC/MS/MS equipped with a turbo-ion spray source. Compound I and N-(2-(5-chloropyridin-2-ylcarbaomoyl)-4-methoxyphenyl)-4-(N,N-dimethylcarbamimidoyl)-2-fluorobenzamide were separated on a Thermo Hypersil-Keystone Betasil C₁₈ column (4.6×100 mm, 5 μm; Fisher Scientific, Houston, Tex.). A mobile phase gradient mixture of 90% mobile phase A (0.5% formic acid in water) and 10% mobile phase B (0.5% formic acid in 90% acetonitrile) to 40% mobile phase B (programmed over 2.8 minutes). The peak areas of the m/z 411→250 product ion (Compound I) were measured against those of the m/z 470→342 product ion (N-(2-(5-chloropyridin-2-ylcarbaomoyl)-4-methoxyphenyl)-4-(N,N-dimethylcarbamimidoyl)-2-fluorobenzamide) in positive ion mode. The analytical range was 0.500 to 10,000 ng/mL.

### Data Analysis

Sample Compound I concentrations below the lower limit of quantitation (LLQ) were reported as <0.500 ng/mL. These values were treated as zero for pharmacokinetic calculations.

Compound I pharmacokinetic parameter values were calculated by noncompartmental analysis of the plasma concentration-time data using Watson LIMS software (version 7.1). Terminal elimination rate constant (k) was calculated as the absolute value of the slope of linear regression of the natural logarithm (ln) of plasma concentration versus time during the terminal phase of the plasma concentration-time profile. Apparent terminal half-life (T_{1/2}) values were calculated as ln(2)/k. Area under the plasma concentration-time profile (AUC) values were estimated using the linear trapezoidal rule. AUCₐₗₗ values were calculated from time 0 to the time of the last detectable concentration. AUC_{(0-inf)} values were calculated as the sum of the corresponding AUCₐₗₗ and the last detectable concentration divided by k. Systemic clearance (CL) was calculated from IV Dose/AUC_{(0-inf)}. Volume of distribution (Vz) was calculated from IV Dose/[k·AUC_{(0-inf)}]. Volume of distribution at steady-state (Vss) was calculated from CL*Mean Residence Time. Maximum plasma concentrations (Cₘₐₓ) and time to reach Cₘₐₓ (Tₘₐₓ) were recorded as observed. Percentage oral bioavailability was calculated by taking the ratio of dose-normalized AUC_{(0-inf)} values (AUC/D) following PO and IV administration. The results are shown in Tables 1 and 2.

TABLE 1
Pharmacokinetic parameters of Compound I in rat, dog, and monkey after
intravenous administration determined by noncompartmental analysis

| | | Mean ± SD | | |
|---|---|---|---|---|
| Parameter | Unit | Rat | Dog | Monkey |
| Dose | mg/kg | 1 | 1 | 1 |
| T_{1/2} | hr | 2.86 ± 1.40 | | |
| AUCₐₗₗ | ng*hr/mL | 5376 ± 1186 | 1615 ± 360 | 12550 ± 5995 |
| AUC_{(0-inf)} | ng*hr/mL | 5404 ± 1163 | 1622 ± 363 | 12560 ± 5998 |
| Vz | L/kg | 0.757 ± 0.328 | 2.73 ± 2.45 | 2.31 ± 1.71 |
| CL | mL/min/kg | 3.19 ± 0.734 | 10.7 ± 2.69 | 1.66 ± 1.06 |
| Vss | L/kg | 0.368 ± 0.026 | 0.843 ± 0.288 | 0.353 ± 0.059 |
| Dose excreted unchanged in | % | 0.248 ± 0.019 | | |
| urine | | | | |

Noncompartmental analysis was performed using Watson LIMS software (version 7.1).
T_{1/2}: Terminal half-life
AUC: Area under the plasma concentration vs. time curve
Vz: Volume of distribution
CL: Systemic clearance
Vss: Volume of distribution at steady-state

TABLE 2
Pharmacokinetic parameters of Compound I in rat, dog, and monkey
after oral administration determined by noncompartmental analysis

| | | Mean ± SD | | |
|---|---|---|---|---|
| Parameter | Unit | Rat | Dog | Monkey |
| Dose | mg/kg | 10 | 5 | 5 |
| T_{1/2} | hr | 2.72 ± 0.29 | | |
| Tₘₐₓ | hr | 0.250 ± 0.00 | 0.583 ± 0.382 | 2.00 ± 0.00 |
| Cₘₐₓ | ng/mL | 28890 ± 2084 | 2717 ± 474 | 6041 ± 1877 |
| AUCₐₗₗ | ng*hr/mL | 68510 ± 12510 | 5464 ± 1471 | 42140 ± 17240 |
| AUC_{(0-inf)} | ng*hr/mL | 68590 ± 12490 | 5475 ± 1475 | 42150 ± 17250 |
| AUC/D | kg*hr/mL | 6859 ± 1249 | 1095 ± 295 | 8430 ± 3449 |
| F | % | 127 ± 23.1 | 68.5 ± 15.5 | 71.6 ± 18.1 |

Noncompartmental analysis was performed using Watson LIMS software (version 7.1).
T_{1/2}: Terminal half-life
Tₘₐₓ: Time to reach maximal plasma concentration
Cₘₐₓ: Maximal plasma concentration
AUC: Area under the plasma concentration vs. time curve
% F: Absolute bioavailability

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

What is claimed is:

1. A crystalline form of a mesylate salt of the compound of 5-chloro-N-((1-(4-(2-oxopyridin-1(2H)-yl)phenyl)-1H-imidazol-4-yl)methyl)thiophene-2-carboxamide, wherein the crystalline form is characterized by an X-ray powder diffraction (XRPD) pattern substantially the same as:
(a) an X-ray powder diffraction (XRPD) pattern having at least six 2θ° peaks selected from the group consisting of about 8.8, about 10.6, about 13.8, about 17.1, about 18.5, about 21.3, about 23.5, about 25.2, about 27.9 and about 29.0; or
(b) an X-ray powder diffraction (XRPD) pattern having at least six 2θ° peaks selected from the group consisting of about 8.5, about 10.4, about 16.6, about 17.1, about 17.8, about 22.7, about 23.5, about 24.5, about 26.9 and about 27.5.

2. The crystalline form of claim 1 characterized by an X-ray powder diffraction pattern having at least six 2θ° peaks selected from the group consisting of about 8.8, about 10.6, about 13.8, about 17.1, about 18.5, about 21.3, about 23.5, about 25.2, about 27.9 and about 29.0.

3. The crystalline form of claim 1 characterized by an X-ray powder diffraction pattern having at least eight 2θ° peaks selected from the group consisting of about 8.8, about 10.6, about 13.8, about 17.1, about 18.5, about 21.3, about 23.5, about 25.2, about 27.9 and about 29.0.

4. The crystalline form of claim 1 characterized by an X-ray powder diffraction pattern substantially the X-ray powder diffraction pattern as FIG. 3.

5. The crystalline form of claim 1, characterized by an X-ray powder diffraction pattern having at least six 2θ° peaks selected from the group consisting of about 8.5, about 10.4, about 16.6, about 17.1, about 17.8, about 22.7, about 23.5, about 24.5, about 26.9 and about 27.5.

6. The crystalline form of claim 1, characterized by an X-ray powder diffraction pattern having at least eight 2θ° peaks selected from the group consisting of about 8.5, about 10.4, about 16.6, about 17.1, about 17.8, about 22.7, about 23.5, about 24.5, about 26.9 and about 27.5.

7. The crystalline form of claim 1 characterized by an X-ray powder diffraction pattern substantially the X-ray powder diffraction pattern as FIG. 4.

8. The crystalline form of claim 1, comprising a hydrate of the salt.

9. A mesylate salt of the compound of 5-chloro-N-((1-(4-(2-oxopyridin-1(2H)-yl)phenyl)-1H-imidazol-4-yl)methyl)thiophene-2-carboxamide, wherein at least a portion of the salt is in a crystalline form characterized by an X-ray powder diffraction (XRPD) pattern substantially the same as
(a) an X-ray powder diffraction (XRPD) pattern having at least four 2θ° peaks selected from the group consisting of about 8.8, about 10.6, about 13.8, about 17.1, about 18.5, about 21.3, about 23.5, about 25.2, about 27.9 and about 29.0; or
(b) an X-ray powder diffraction (XRPD) pattern having at least four 2θ° peaks selected from the group consisting of about 8.5, about 10.4, about 16.6, about 17.1, about 17.8, about 22.7, about 23.5, about 24.5, about 26.9 and about 27.5;
or combinations thereof.

10. A composition comprising the crystalline form of any one of claims 1 to 8 or the salt of claim 9, and a pharmaceutically acceptable carrier.

11. A method for preventing or treating a condition in a mammal characterized by undesired thrombosis comprising administering to said mammal a therapeutically effective amount of the crystalline form of any one of claims 1 to 8 or the salt of claim 9.

12. A method for prevention of stroke in atrial fibrillation patients; prevention of thrombosis in medically ill patients; prevention and treatment of deep vein thrombosis; prevention of arterial thrombosis in acute coronary syndrome patients; and/or secondary prevention of myocardial infarction, stroke or other thrombotic events in patients who have had a prior event, comprising administering to said mammal a therapeutically effective amount of the crystalline form of any one of claims 1 to 8 or the salt of claim 9.

13. A method for inhibiting the coagulation of a blood sample comprising the step of contacting said sample with the crystalline form of any one of claims 1 to 8 or the salt of claim 9.

14. A composition which is a free-flowing dosable aqueous suspension, wherein the composition comprises a pharmaceutically acceptable aqueous carrier and a mesylate salt of the compound of 5-chloro-N-((1-(4-(2-oxopyridin-1(2H)-yl)phenyl)-1H-imidazol-4-yl)methyl)thiophene-2-carboxamide; wherein the concentration of the compound is at least 40 mg/mL, and wherein at least a portion of the salt is in a crystalline form characterized by an X-ray powder diffraction (XRPD) pattern substantially the same as:
(a) an X-ray powder diffraction (XRPD) pattern having at least four 2θ° peaks selected from the group consisting of about 8.8, about 10.6, about 13.8, about 17.1, about 18.5, about 21.3, about 23.5, about 25.2, about 27.9 and about 29.0; or
(b) an X-ray powder diffraction (XRPD) pattern having at least four 2θ° peaks selected from the group consisting of about 8.5, about 10.4, about 16.6, about 17.1, about 17.8, about 22.7, about 23.5, about 24.5, about 26.9 and about 27.5;
or is in an amorphous form,
or combinations thereof.

15. The composition of claim 13, wherein the concentration of the compound is up to about 100 mg/mL.

16. The composition of claim 13, wherein the crystalline form is characterized by an X-ray powder diffraction pattern substantially the same as provided in FIG. 3 or FIG. 4.

17. A method of preparing the composition of claim 14, which method comprises mixing the mesylate salt of 5-chloro-N-((1-(4-(2-oxopyridin-1(2H)-yl)phenyl)-1H-imidazol-4-yl)methyl)thiophene-2-carboxamide and the pharmaceutically acceptable aqueous carrier, and storing the mixture at a temperature of about or below 8° C. for a period of time sufficient to form the composition.

18. The method of claim 17 wherein the temperature is from about 2° C. to about 8° C.

19. The method of claim 17 wherein the temperature is sufficient to cause the mixture to freeze and wherein the method further comprises thawing the frozen mixture.
